(19) United States
(12) Patent Application Publication
(10) Pub. No.: US 2011/0152841 A1
(43) Pub. Date: Jun. 23, 2011

(54) MEDICAL CONNECTOR DEVICE

(76) Inventor: Shigeru Nemoto, Tokyo (JP)

(21) Appl. No.: 13/055,914

(22) PCT Filed: Jul. 21, 2009
(86) PCT No.: PCT/JP2009/063056

§ 371 Date: Jan. 25, 2011

(30) Foreign Application Priority Data
Jul. 25, 2008 (JP) 2008-192163
Jan. 21, 2009 (JP) 2009-011295

(51) Int. Cl. A61M 39/10

(52) U.S. Cl. 604/533

(57) ABSTRACT

To provide a medical connector device capable of preventing a backflow of liquid from a catheter and ensuring excellent watertight sealing in a connector. The medical connector device includes a catheter (2), a catheter hub (30) having a leading end portion supporting a base portion of the catheter (2), a check valve (45) placed within the hub (30), and a male connector (10) having a male lure portion (15) to be inserted into the hub (30). The male connector (10) includes a leading conduit (16) thinner than the lure portion (15) at a leading end of the lure portion. When the male connector (10) is not being connected to the catheter hub (30), the check valve (45) prevents a backflow of liquid. When the male connector (10) is being connected to the catheter hub (30), the leading conduit (16) is inserted into a central hole (45h) to increase the diameter of the central hole (45h) to allow a flow of liquid, and the check valve (45h) provides watertight sealing between an outer peripheral face of the leading conduit (16) and an inner peripheral face of the hub (30).

### TECHNICAL FIELD

The present invention relates to a medical connector device for connecting, for example, a tube to an indwelling needle, more particularly, to a medical connector device capable of preventing a backflow of liquid from a catheter and ensuring excellent watertight sealing in the connector.

### BACKGROUND ART

Conventionally employed medical imaging diagnostic apparatuses include CT (Computed Tomography) scanners, MRI (Magnetic Resonance Imaging) apparatuses, PET (Positron Emission Tomography) apparatuses, ultrasonic diagnostic apparatuses, CT angiography apparatuses, MR angiography apparatuses and the like.

When the abovementioned apparatuses are used, a contrast medium or physiological saline may be injected into a patient's body in order to provide clearer diagnostic images, by way of example.

For injecting a chemical liquid into a patient, for example, a catheter of an indwelling needle is first inserted into a blood vessel of an arm of the patient in order to be ready to inject the chemical liquid within a syringe into the patient. Then, a piston of the syringe is pushed to push out and inject the chemical liquid from the syringe into the patient's body through a tube and the catheter. Automatic injectors for automatically performing the chemical liquid injection have conventionally been known.

The indwelling needle typically includes a catheter which is inserted into a patient's body and a catheter hub which supports the catheter. A lure lock method (see, for example, Patent Document 1) is known by way of example as a structure for connecting the catheter hub to a chemical liquid tube.

### LIST OF DOCUMENTS

Patent Document 1: Utility Model Application No. S62-116740

### SUMMARY OF INVENTION

For example, in injecting a contrast medium into a patient as described above, the chemical liquid is at a higher pressure than that in normal intravenous drip. Thus, a connector used therefore needs to have excellent watertight sealing which prevents leaks even when the chemical liquid is at a high pressure.

On the other hand, it is desired that the indwelling needle is formed to prevent a backflow of blood through the catheter when the catheter is inserted into a patient's blood vessel.

The present invention has been made in view of the abovementioned problems, and it is an object thereof to provide a medical connector device capable of preventing a backflow of liquid from a catheter and ensuring excellent watertight sealing in the connector.

To achieve the abovementioned object, a medical connector device according to the present invention includes:

a catheter inserted into a patient's body;

a catheter hub having a generally tubular shape as a whole and having a base end portion opened and a leading end portion supporting a base portion of the catheter;

a check valve having a central hole with an increasable and reducible diameter and placed within the catheter hub; and

a male connector having a male lure portion to be inserted into the catheter hub from its opened side and connected to the catheter hub,

wherein the male connector includes a leading conduit at a leading end of the male lure portion, the conduit being thinner than the male lure portion

when the male connector is not being connected to the catheter hub, the diameter of the central hole of the check valve is reduced to prevent a backflow of liquid from the catheter, and

when the male connector is being connected to the catheter hub, the leading conduit is inserted into the central hole to increase the diameter of the central hole to allow a flow of liquid, and the check valve provides watertight sealing between an outer peripheral face of the leading conduit and an inner peripheral face of the catheter hub in a state.

According to the configuration, since the check valve is provided within the catheter hub, a chemical liquid from the catheter does not flow reversely to leak in the state in which the male connector is not connected to the catheter hub. In the state in which the male connector is connected to the catheter hub, the check valve serves as a seal member, so that sufficient watertight sealing is ensured.

In the present invention, the leading conduit of the male connector may have a tapered shape. The male lure portion and the leading conduit may be formed of different members.

When the check valve further includes a tapered face which guides the leading conduit to the central hole, the leading conduit of the male connector is advantageously inserted easily into the central hole.

The medical connector device according to the present invention may further include fixing means for fixing the male connector to the catheter hub,

wherein the fixing means includes:

a plurality of engaging spirals formed on an outer peripheral face of the catheter hub; and

a plurality of protrusions formed on the male connector and engageable with the engaging spirals, and

wherein the male connector is screwed into the catheter hub and the

male connector is rotated within a range of 180° or smaller about an axis line to engage the protrusion with the engaging spiral in order to connect the male connector to the catheter hub.

The medical connector device according to the present invention further includes fixing means for fixing the male connector to the catheter hub,

wherein the fixing means includes:

a engaging pin formed on one of the male connector and the catheter hub and extending outward in a generally diameter direction; and

a engaging groove of generally spiral shape formed in a tubular portion of the other of the male connector and the catheter hub, the engaging pin being slid within the engaging groove,

wherein the engaging pin engages at the end of the groove to connect the male connector to the catheter hub.

A medical connector device according to another aspect of the present invention further includes a bridging portion which connects a leading end of the engaging pin and a portion of one of the male connector and the catheter hub, in an axis direction, to form an engaging hole,

wherein a portion of the tubular portion of the other of the male connector and the catheter hub is inserted into and engages with the engaging hole.

According to the configuration, since the male connector is connected to the catheter hub, for example in the state in which the portion of the tubular portion of the male connector engages with the engaging hole, the reliability of the connection is further improved.

In another aspect of the present invention, the engaging pin includes two engaging pins formed on an outer periphery of one of the male connector and the catheter hub, and the bridging portion is provided for only one of the two engaging pins.

According to the configuration, the bridging portion is provided for only one of the engaging pines to form an asymmetric shape, so that the top and the bottom (by way of example) of the catheter hub can be identified by using the connecting portion as a mark.

In another aspect of the present invention, the fixing means includes:

a protrusion formed within the engaging hole; and

a recess formed in the tubular portion of the other of the male connector and the catheter hub, the protrusion engaging with the recess.

Thus, in the configuration in which not only the engaging operation of the engaging groove and the engaging pin but also the engaging operation of the protrusion (for example, a rib) and the recess can be used to fix the male connector to the catheter hub, the fixing of those members can be performed more reliably.

As described above, according to the present invention, the medical connector device is provided in which the function of the check valve placed within the catheter hub can prevent a backflow of liquid from the catheter and can ensure excellent watertight sealing in the connector.

### BRIEF DESCRIPTION OF THE DRAWINGS

FIG. 1 is a section view of a medical connector device according to an embodiment of the present invention.

FIG. 2 is a section view showing a catheter hub of the connector device in FIG. 1.

FIG. 3 is a section view showing a male connector of the connector device in FIG. 1.

FIG. 4 is a diagram showing a engaging spiral at a base end of the catheter hub.

FIG. 5 is a section view showing another example of a check valve.

FIG. 6 is a section view showing yet another example of the check valve.

FIG. 7A is a section view showing another example of the male connector.

FIG. 7B is a section view showing another example of the catheter hub.

FIG. 8 is a perspective view showing the outer appearance of a medical connector device according to Embodiment 2 (connection state).

FIG. 9 is a perspective view showing the connector device in FIG. 8 before connection.

FIG. 10 is a plan view (a) and a side view (b) representing the state in FIG. 8.

FIG. 11 is a plan view representing the state in FIG. 9.

FIG. 12 is a rear view showing a catheter hub viewed from a base end side.

FIG. 13 is a front view showing a male lure portion of a male connector viewed from a leading end side.

FIG. 14 is a plan view showing another example of the medical connector device according to Embodiment 2.

FIG. 15 is a plan view showing yet another example of the present invention.

FIG. 16 is a perspective view showing a connector device according to another embodiment before connection.

FIG. 17 is a perspective view showing only a male connector in FIG. 16.

FIG. 18 is a perspective view showing only a catheter hub in FIG. 16.

FIG. 19 is a side view of the male connector.

FIG. 20 is a side view of the catheter hub.

FIG. 21 is a diagram for showing the angle of an inner wall of a engaging groove.

FIG. 22 is a perspective view showing a connector device according to yet another embodiment before connection.

FIG. 23 is a plan view (connection state) of the connector device in

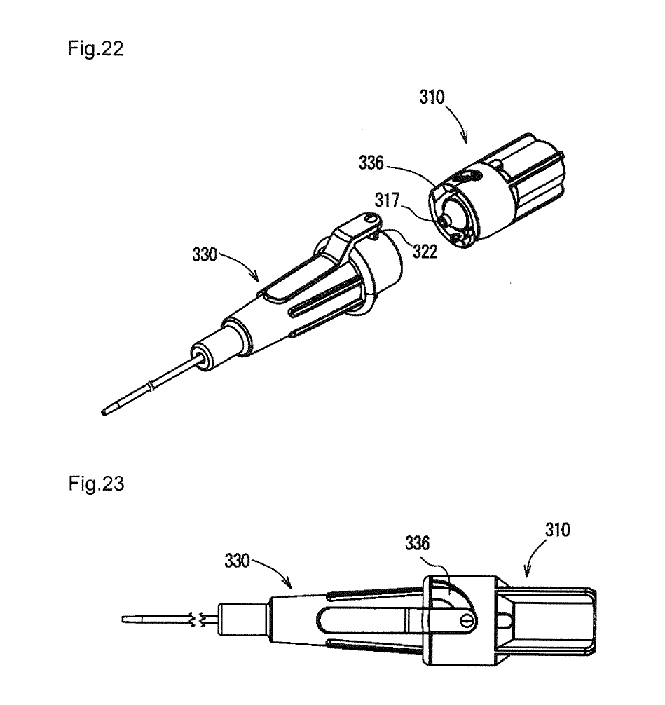

FIG. 22.

FIG. 24 is a section view (connection state) of the connector device in FIG. 22.

FIG. 25 is a section view and a side view of the catheter hub.

FIG. 26 is a diagram showing the catheter hub.

FIG. 27 is a diagram showing the male connector.

FIG. 28 is a diagram for explaining the connection of the connector device.

### BEST MODE FOR CARRYING OUT THE INVENTION

Embodiments of the present invention will hereinafter be made with reference to the accompanying drawings.

### Embodiment 1

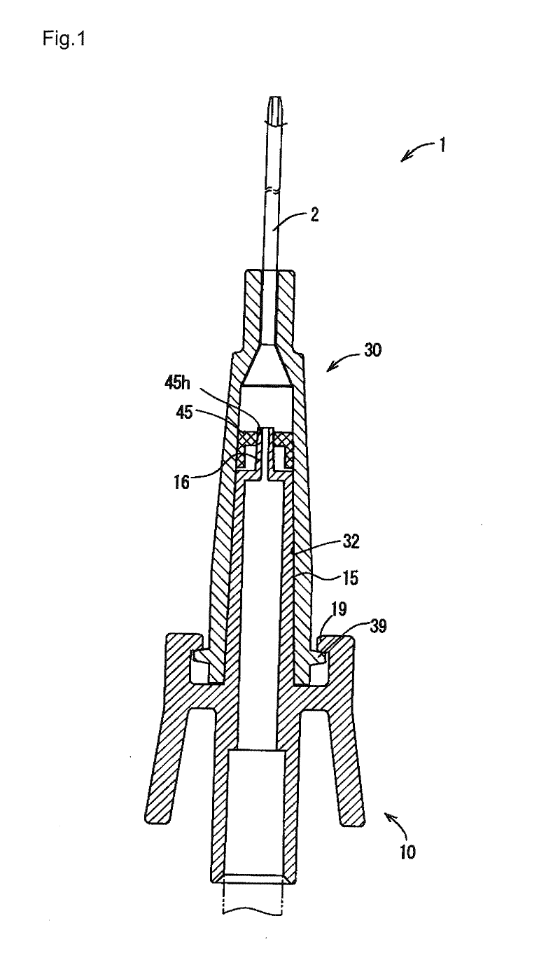

FIG. 1 is a section view of a medical connector device according to an embodiment of the present invention and shows a male connector and a catheter hub. In the following, while the terms of “top,” “bottom,” “left,” and “right” indicating directions are used in accordance with the illustration of the figures, they are not intended to limit the present invention.

As shown in FIG. 1, medical connector device 1 includes catheter hub 30 of generally tubular shape and male connector 10 connected thereto.

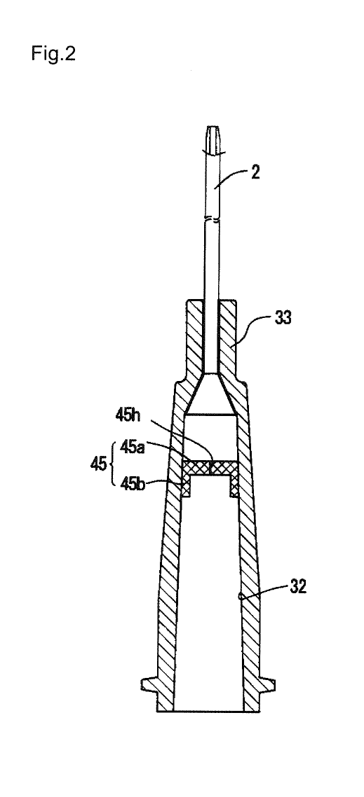

Catheter hub 30 supports a base end of catheter 2 as shown also in FIG. 2 and is made of resin, by way of example. Catheter hub 30 has an internal hole which is opened on a base end side and leads to a leading end side. Catheter hub 30 has tubular portion 33 at leading end for supporting the base end portion of catheter 5. A female lure face (tapered face) 32, gradually narrowed from the opening portion at the base end toward the leading end, is formed inside the catheter hub 30.

A check valve 45 is provided at a position somewhat closer to the leading end than the central portion in a longitudinal direction within catheter hub 30. The material of check valve 45 is an elastic material such as isoprene rubber, for example. In this example, check valve 45 has valve wall 45a of disc shape which extends in a radius direction of catheter hub 30 to close a flow path. The valve 45 has a tubular portion 45b which extends from an outer peripheral edge of the valve wall 45a toward the base end side (downward in FIG. 2). An outer peripheral face of tubular portion 45b is fixed to female lure face 32 within the catheter hub. While the fixing means is not limited particularly, it is possible to use fixing with an adhesive or fixing with ultrasonic welding, for example.

Another example of placement of check valve 45 will be described later with reference to other figures.

Single central hole 45h is formed at the center of valve wall 45a. The Central hole 45h expands or contracts in diameter. When male connector 10 is not connected as shown in FIG. 2, the hole contracts to prevent a backflow of liquid (for example, blood of a patient) from catheter 2. Central hole 45h may be a shape of linear-cut shape, cross-cut shape, or Y-cut shape, for example.

Catheter 2 is conventionally known one, and for example, has flexibility and its leading end is inserted into a patient's blood vessel or the like.

Catheter hub 30 with catheter 2 is called an “indwelling needle”. To insert such flexible catheter 2 smoothly into a patient's blood vessel or the like, a double needle structure including a hard inner needle inside catheter 2 is employed. The double needle structure is conventionally known and is disclosed in Japanese Patent Laid-Open No. 2007-275304, for example. In the typical double needle structure, an inner needle made of metal is slidably inserted into catheter 2 (outer needle), and in an initial state, a leading end of the inner needle slightly protrudes from a leading end of catheter 2. Such a double structure can ensure the rigidity of the whole needle to realize the smooth insertion to a patient's blood vessel or the like. The inner needle is pulled out after the insertion, so that only catheter 2 serving as the outer needle is left. Various proposals have conventionally been made of mechanisms for pulling out the inner needle. The present embodiment can employ a mechanism for removing the inner needle by pulling a wire connected to a base end of the inner needle, or a mechanism for removing the inner needle by using the urging force of a spring, for example.

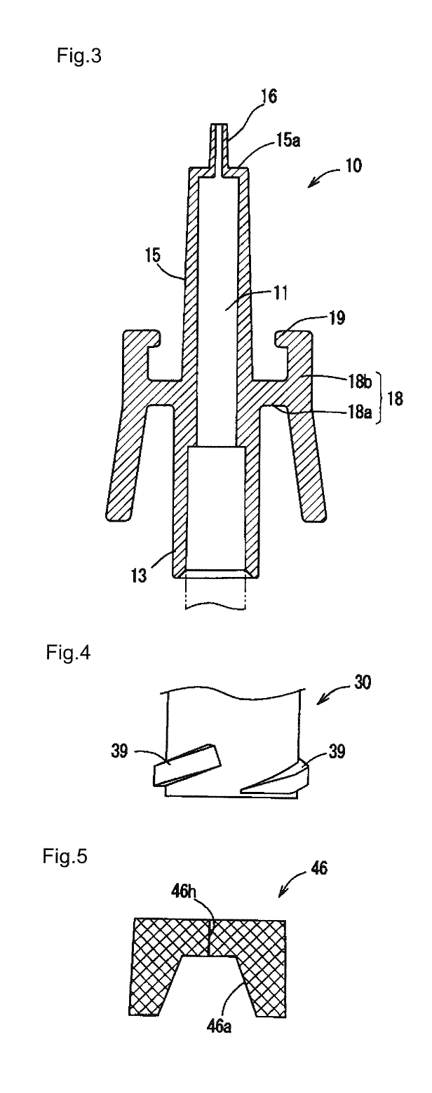

Male connector (see FIG. 3) is a resin component, by way of example, and has liquid path 11 through which a chemical liquid passes. Male connector 10 has barrel portion 13 of straight tube shape in the base end side and male lure portion 15 extending from the barrel portion 13 to a leading end.

A pair of engaging hooks 18 are formed on both sides of left and right of barrel portion 13 for connecting male connector 10 to catheter hub 30.

A chemical liquid tube is connected to an inlet portion of liquid path 11. Chemical liquid is supplied from a syringe, not shown, through the chemical liquid tube.

An outer peripheral face of male lure portion 15 is formed as a tapered face narrowed toward the leading end that conforms to female lure face 32 of catheter hub 30. The taper may be approximately 6° , by way of example.

An end face 15a is formed at the leading end of male lure portion 15. When the male connector 10 is connected to catheter hub 30 (see FIG. 1), the face 15a is positioned in front of check valve 45.

Returning to FIG. 3, a hollow leading conduit 16 thinner than male lure portion 15 is formed on end face 15a at the leading end of male lure portion 15. Leading conduit 16 is inserted into central hole 45a of the check valve, as described later. Thus, an outer peripheral face of leading conduit 16 may be formed in a tapered shape in order to facilitate the insertion.

Each of the pair of engaging hooks 18 has base portion 18a protruding outward in a diameter direction from barrel portion 13 of male connector 10 and lever 18b having an intermediate portion supported by an end of the base portion 18a. Protrusion 19 protruding inward in the diameter direction is formed on the inner side of the leading end of lever 18b.

Protrusions 19 engage to two engaging spirals 39 formed at a base end portion of an outer peripheral face of catheter hub 30 as shown in FIG. 4. Engaging spirals 39 are formed along the outer peripheral face of catheter hub 30 and are placed opposite to each other. Each spirals occupies approximately 180° range of the outer peripheral face.

Next, description will be made of a method of using medical connector device 1 of the present embodiment configured as above.

First, catheter 2 is inserted into a blood vessel of a patient. Specifically, catheter 2 in the double needle state is inserted into the patient's blood vessel similarly to the conventional insertion method and then the inner needle within the catheter is pulled out, leaving only the indwelling needle (catheter 2 and catheter hub 30 which supports it) on the patient's arm. Since central hole 45h of the check valve within catheter hub 30 is closed in this state, blood or the like is not leaked outside.

Next, male connector 10 is connected to catheter hub 30.

By way of example, while an operator grasps and holds catheter hub 30 by one hand, grasps connector 10 by the other hand, and inserts male lure portion 15 of male connector 10 into catheter hub 30. Then, the operator screws male connector 10 into catheter hub 30 (at an rotation angle of 180° or less), and each protrusion 19 of the male connector engages to each engaging spiral 39 on the outer peripheral face of the catheter hub, thereby male connector 10 is connected to hub 30.

When male connector 10 is inserted into catheter hub 30, leading conduit 16 of the male connector pushes and opens central hole 45h of the check valve to pass therethrough (see FIG. 1). This makes it ready to supply the chemical liquid from male connector 10 to catheter 2.

Since check valve 45 made of the elastic body is compressed between the outer peripheral face of leading conduit 16 of the male connector and an inner peripheral face of catheter hub 30 in the connection state shown in FIG. 1, check valve 45 serves as a seal member.

In the configuration of the present embodiment, male connector 10 is connected to catheter hub 30 with the screw-in operation, and the male lure face of male connector 10 is pushed against the female lure face of the catheter hub in the connection state shown in FIG. 1. Therefore both the faces contact intimately, thereby sufficient watertight sealing is obtained.

While the leading conduit 16 of the male connector protrudes from an end face of check valve 45 in the state in which male connector 10 is connected to catheter hub 30 in the example of FIG. 1, the present invention is not limited thereto.

To inject a contrast medium or the like into the patient, an injector, not shown, may be operated in the state in which male connector 10 is connected to catheter hub 30 as shown in FIG. 1 (by way of example). Then, the contrast medium or the like within the syringe is supplied to male connector 10 through the chemical liquid tube (not shown). It passes therethrough and flows out of the leading conduit 16, and flows into catheter hub 30 and then injected into the patient's body through catheter 2.

After a series of injection steps is completed, both of male connector 10 and catheter hub 30 may be pulled to remove catheter 2 from the patient's body. Alternatively, male connector 10 may be first removed from hub 30 and then hub 30 to remove catheter 5. According to the configuration of the present embodiment, even when male connector 10 is first removed blood or the like is not leaked since the check valve 45 is provided in the catheter hub 30

As described above, according to medical connector device 1 of the present embodiment, the check valve 45 prevents a backflow of blood from catheter 2 in the state in which male connector 10 is not connected, and in the state in which male connector 10 is connected, check valve 45 serves as the seal member to ensure excellent watertight sealing.

Especially, since check valve 45 serves as the seal member, any extra component such as an O-ring for watertight sealing can be omitted and device 1 is advantageous in simplifying the configuration.

While an embodiment of the present invention has been described, the present invention is not limited thereto.

For example, hub 30 having two engaging spirals 39 has been described in FIG. 4, the number of engaging spirals 39 is not limited to two, and four or six engaging spirals may be provided. The rotation angle for connecting the male connector is approximately 90° for four engaging spirals, or is approximately 60° for six engaging spirals.

FIG. 5 is a section view showing another example of the check valve. Check valve 46 has tapered face 46a narrowed toward central hole 46h for guiding leading conduit 16 of the male connector (see FIG. 1) toward central hole 46h. Such a check valve has the advantage that leading conduit 16 of the male connector is easily inserted into central hole 46h in connecting the male connector.

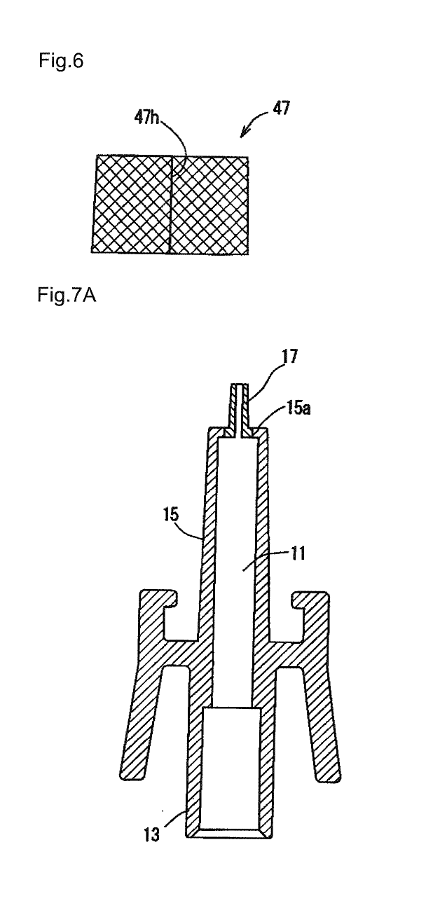

FIG. 6 is a section view showing yet another example of the check valve. Check valve 47 has a short column shape and has single central hole 47h at the center. The check valve may have a relatively simple shape as shown.

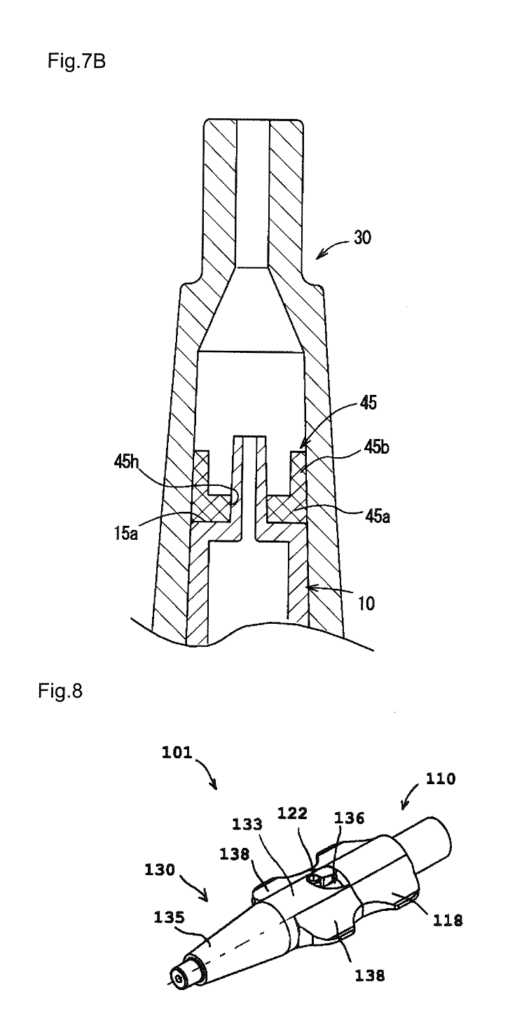

FIG. 7A is a section view showing another example of the male connector. In the male connector, leading conduit 17 at the leading end is formed of a member different from another part (male lure portion 15). According to the configuration characteristic (for example, rigidity) of the leading conduit 17 can be selected accordingly.

FIG. 7B is a section view showing another example of the catheter hub.

As shown in FIG. 7B, check valve 45 may be placed upside down relative to the example show in FIG. 2. In this example, valve wall 45a of check valve 45 is located on a lower side in FIG. 7A in catheter hub 30. Although not limited particularly, in the state in which male connector 10 is connected to catheter hub 30, leading end face 15a of male connector 10 may be pushed against and is brought into intimate contact with a face of valve wall 45a located on the lower side. As described above, central hole 45h may be a shape of linear-cut shape, cross-cut shape, or Y-cut shape, for example.

### Embodiment 2

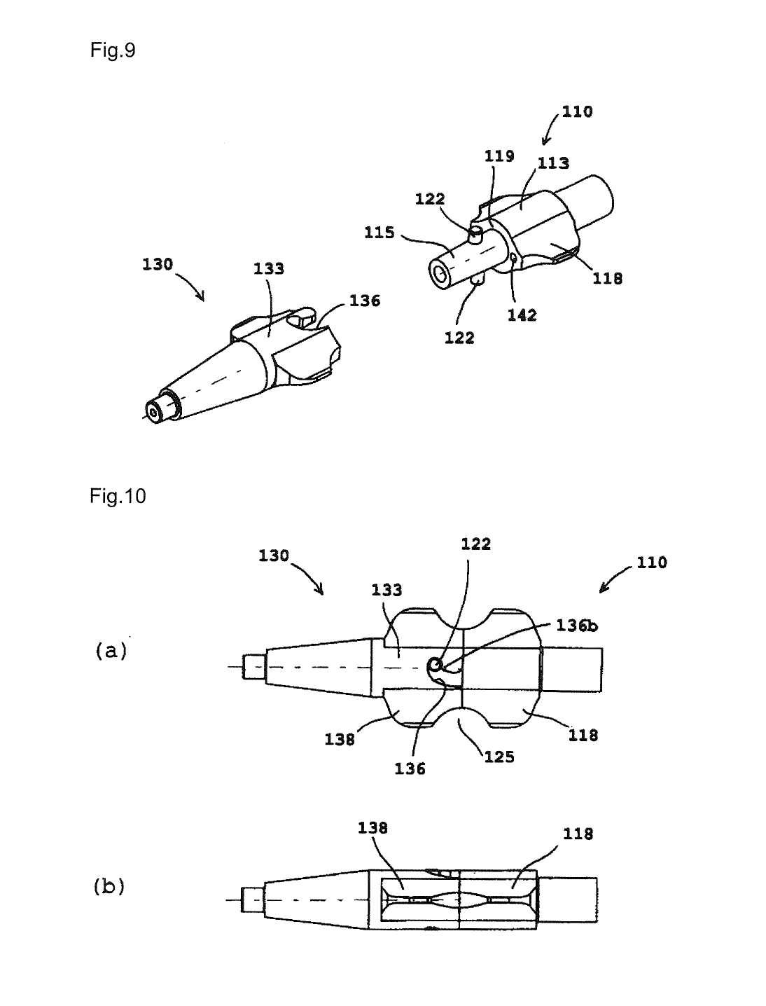

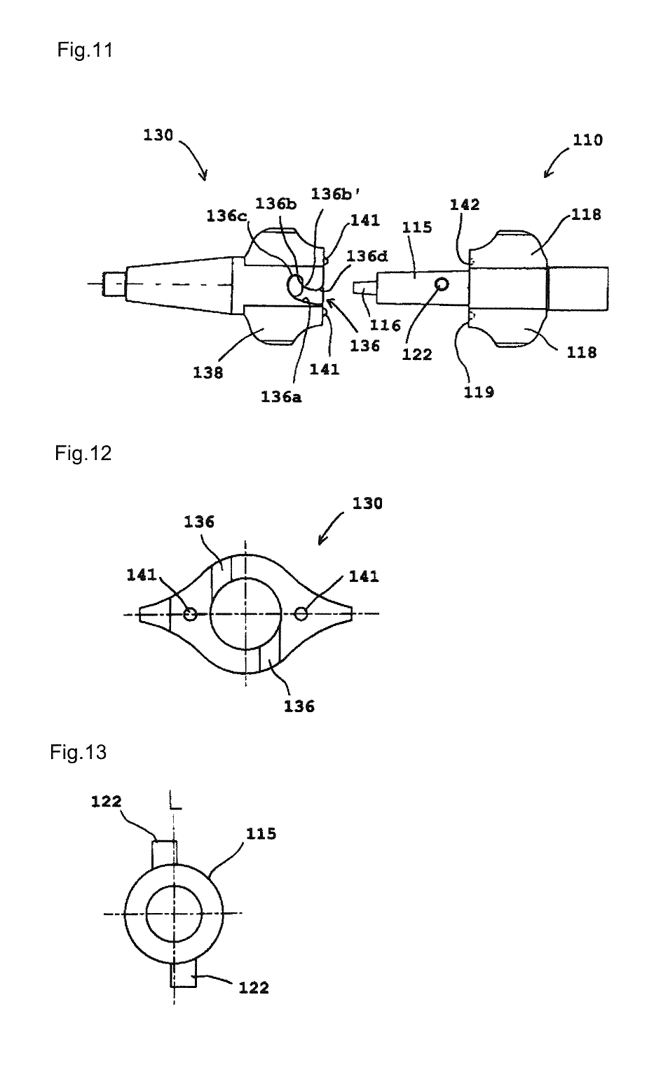

A modification example of the connection structure of the male connector and the catheter hub will be described with reference to FIGS. 8 to 13. FIG. 8 is a perspective view showing the outer appearance of a medical connector device of the present embodiment (connection state). FIG. 9 is a perspective view showing the outer appearance before connection. FIG. 10(a) is a plan view and FIG. 10(b) is a side view representing the state in FIG. 8. FIG. 11 is a plan view representing the state in FIG. 9. FIG. 12 is a rear view of a catheter hub viewed from its base end side. FIG. 13 is a front view of a male lure portion of a male connector viewed from a leading end side.

As shown in FIGS. 8 and 9, connector device 101 includes catheter hub 130 and male connector 110 connected thereto.

Catheter hub 130 includes barrel portion 133 of tubular shape on a base end side and tapered portion 135 extending from barrel portion 133 to a leading end side and narrowed toward the end. Fin portions 138, 138 are formed on both sides of left and right of barrel portion 133 to protrude outward in a diameter direction. A catheter (not shown) is connected to the leading end of tapered portion 135.

As shown in FIGS. 8 to 10, engaging groove 136 of generally spiral shape is formed at the base end of barrel portion 133 for connecting hub 130 to male connector 110. As described later, engaging pin 122 formed on male connector 110 is inserted into and engages to inside of groove 136, to connect the male connector to the catheter hub. As shown in FIG. 12, engaging groove 136 is provided for each on an upper side and a lower side of hub 130. One engaging groove 136 has a shape provided by rotating the other engaging groove 136 180° about an axis line.

As shown in FIG. 11, engaging groove 136 has guide face 136a cut from the base end toward the leading end side of hub 130 and gently curved, R surface 136c connected to a leading end of guide face 136a, and straight face 136b connected to an end portion of R surface 136c. R portion 136d is formed at an inlet portion of groove 136 for easily inserting engaging pin 122 (later detailed in detail) into groove 136.

Straight face 136b is formed in a direction orthogonal to the axis line of hub 130. With such straight face 136b formed, even when a force in a tensile direction is applied to male connector 110 in the connection state (see FIG. 10) and engaging pin 122 is pushed against straight face 136b, a force in a rotation direction is not produced and thus the connection of the connector is not loosened easily.

As shown in FIGS. 11 and 12, two small protrusions 141 of hemispherical shape are formed on a base end face of hub 130. These protrusions 141 are placed on both sides of left and right of a hub internal hole. They engage in recesses 142 (see FIG. 9, later described in detail) of the male connector to serve to provide a click feeling at the time of connection.

Although not shown, check valve 45 (see FIG. 1) is placed within male connector 130 as in the abovementioned embodiment. Naturally, the check valve may be one as shown in FIG. 5 or FIG. 6.

As shown in FIG. 9, male connector 110 has barrel portion 113 on a base end side and male lure portion 115 extending therefrom as in the abovementioned embodiment. Fin portions 118 are formed on both sides of left and right of barrel portion 113. Fin portions 118 are the same as ones of the catheter hub 130. Male lure portion 115 is formed to have a tapered face narrowed toward a leading end (for example, 6°) as in Embodiment 1.

Recess 142 of hemispherical shape (by way of example) in which the protrusion 141 engage is formed in front end face 119 of barrel portion 113. While only one is seen in FIG. 9, recess 142 is formed on each of left and right sides of male lure portion 115. As shown in FIG. 11, leading conduit 116 is formed at the leading end of male lure portion 115 as in the abovementioned embodiment (see FIG. 3). Leading conduit 116 is inserted into a central hole of the check valve (not shown) placed within hub 130.

As shown in FIG. 13, engaging pins 122 which are inserted into grooves 136, 136 of the hub and engage therein are formed at the top and the bottom on an outer peripheral face of male lure portion 115. Engaging pin 122 has a cylindrical shape and extends in a direction parallel with vertical central line L. Engaging pins 122 are placed at positions shifted to the left or right from vertical central line L of male lure portion 115 to align with grooves 136 of the hub.

Description will be made of a connection method of the medical connector device of the present embodiment configured as above.

First, a catheter (not shown) attached to the leading end of hub 130 is inserted into a patient's blood vessel.

Next, male connector 110 is connected to catheter hub 130.

By way of example, while an operator grasps fin portions 138, 138 of hub 130 by one hand to hold hub 130 and grasps connector 110 by the other hand to insert male lure portion 115 into hub 130. At this point, male connector 110 is positioned to be somewhat rotated about the axis line so as to allow insertion of engaging pin 122 of male connector 110 into engaging groove 136 of the hub.

As male connector 110 is inserted, an outer peripheral face of engaging pin 122 slides on guide face 136a of groove 136, and engaging pin 122 is guided and moved along guide face 136a. Thus, the overall male connector 110 is screwed into hub 130.

At the time when engaging pin 122 is inserted into a point falling slightly short of the deepest portion of groove 136 (to the extent that engaging pin 122 does not reach straight face 136b at the back of groove 136), a top portion of each protrusion 141 on the base end face of the hub abuts on front end face 119 of male connector 110.

Then, male connector 110 is further screwed to move engaging pin 122 to the deepest portion (final engaging position) of groove 136 to connect male connector 110 and hub 130. Specifically, as male connector 110 is screwed, the top portion of each protrusion 141 slides on front end face 119. Engaging pin 122 moves over corner portion 136b′ before straight face 136b within groove 136 and reaches straight face 136b which is the final engaging position. Substantially simultaneously with engaging pin 122 moving over corner portion 136b′, each protrusion 141 of the hub fits into each recess 142 of male connector 110. The click feeling can be provided at this point.

When male connector 110 is connected to hub 130 through the abovementioned steps, fin portions 118 of the connector and fin portions 138 of the hub are aligned in the axis line direction as shown in FIG. 8. In such a configuration, the operator can check whether the connection is achieved properly or not by seeing whether the fin portions 118 and 138 are aligned or not.

Since fin portions 118 and 138 protrude in the left and right directions (that is, along the skin surface of the patient) and connector device 101 has a flat shape as a whole, connector device 101 can be fixed easily onto the skin of the patient.

For releasing the connection, the operation described above may be reversed. In the present embodiment, since notch portion 125 (of semicircular shape, by way of example) is formed between fin portions 118 and 138 as shown in FIG. 10, fin portions 118 and 138 are grasped more easily to facilitate the removal of male connector 110.

As described above, protrusion 141 of the hub is fitted into recess 142 of the male connector in the connection state (FIG. 8). Thus, to remove (screw) male connector 110, a force in a rotation direction is needed for causing each protrusion 141 to come out of each recess 142. In such a configuration, for example even when some rotation force in a removal direction is applied to male connector 110 in the connection state of the connector (see FIG. 8), male connector 110 is not rotated unless protrusion 141 comes out of recess 142, and the configuration has the advantage that it is not easily loosened.

The abovementioned connection structure is applicable to various other connectors. For example, it is possible to provide a medical connector device in which the check valve is not included within catheter hub 130. In addition, a component corresponding to engaging pin 122 may be provided for hub 130 and a component corresponding to engaging groove 136 may be provided for male connector 110 without being limited to the abovementioned structure. Similarly, a component corresponding to recess 142 may be provided for hub 130 and a component corresponding to protrusion 141 may be provided for male connector 110.

As described above, according to the present embodiment, hub 130 and male connector 110 are connected to each other by the operation of engaging groove 136 and engaging pin 122, and in the connection state, the outer peripheral face of male lure portion 115 of male connector 110 is brought into intimate contact with an inner peripheral face of the internal hole of the hub, so that excellent watertight sealing can be achieved. In addition, since the click feeling can be provided at the time of connection by the operation of protrusion 141 and recess 142, proper connection to the patient can be checked easily. Protrusion 141 and recess 142 also serve as means for preventing the rotation of male connector 110 relative to hub 130 during connection. Thus, even when an unexpected rotation force is applied to male connector 110 or hub 130, male connector 110 is not removed from hub 130 and the connection state is maintained favorably.

While the above description has been made with the example where protrusion 141 and recess 142 for providing the click feeling are provided on both sides of left and right across the axis line of the connector, the present invention is not limited thereto. Protrusion 141 and recess 142 may be provided at two points on the top and the bottom across the axis line of the connector, not on the left and the right. In addition, the number of protrusions 141 and recesses 142 is not limited to two and can be changed as appropriate.

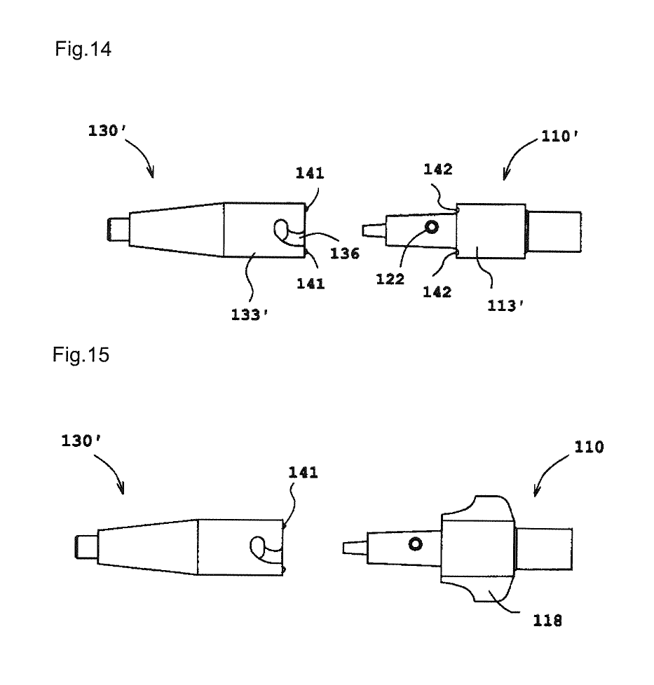

As shown in FIG. 14, the medical connector device of the abovementioned embodiment can be configured such that fin portions 138 and 118 (see, for example FIG. 11) are omitted. In the example of FIG. 14, catheter hub 130′ has barrel portion 133′ of tubular shape and male connector 110′ has barrel portion 113′ of tubular shape. Either of barrel portions 133′, 113′ is not provided with any fin portion. The remaining configuration is identical to that of the abovementioned embodiment described with reference to FIGS. 8 to 13. Such a configuration can realize the operation and effect similar to those in the above embodiment such as the favorable connection of hub 130′ and male connector 110′ with engaging groove 136 and engaging pin 122 and the click feeling at the time of connection obtained through the operation of protrusion 141 and recess 142 provided for hub 130′ and male connector 110′.

As shown in FIG. 15, male connector 110 having fin portion 118 may be connected to catheter hub 130′ having no fin portion.

### Other Embodiment 1

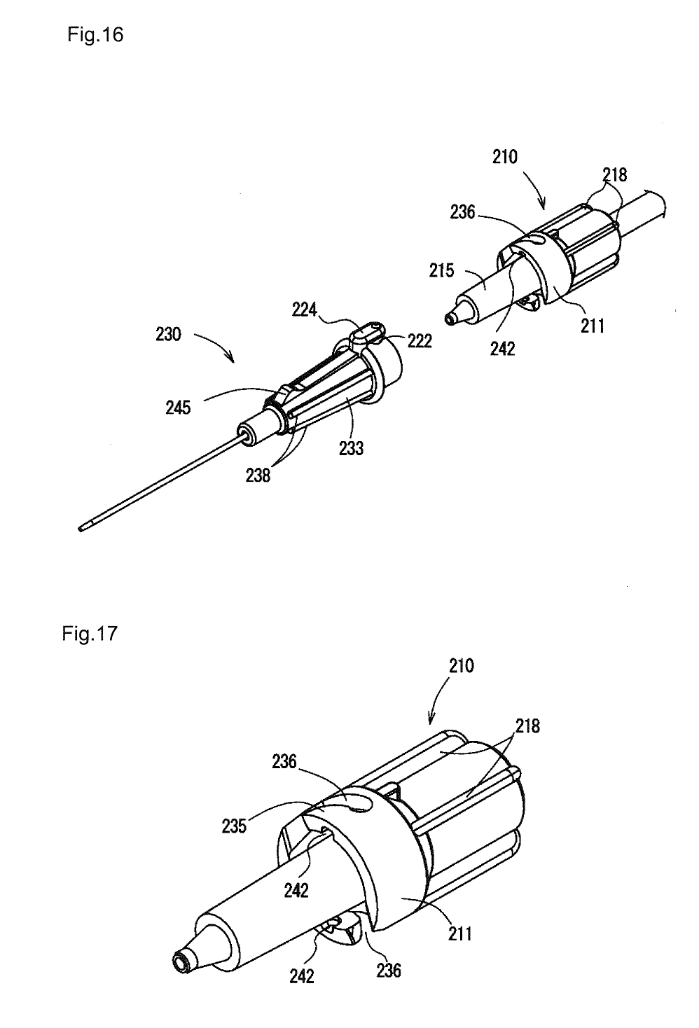

FIGS. 16 to 21 show another modification example of the connection structure of the male connector and the catheter hub.

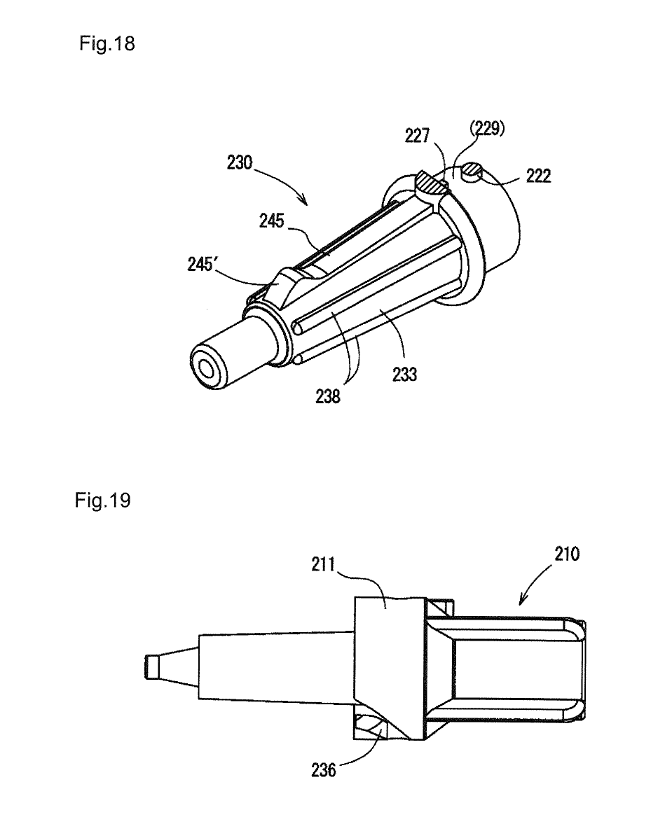

FIG. 16 is a perspective view showing a connector device of the present embodiment before connection. FIG. 17 is a perspective view showing only a male connector. FIG. 18 is a perspective view showing only a catheter hub. In FIG. 18, the illustration of a catheter is omitted, and the illustration of a leading end side of engaging pin 222 (described below in detail) is omitted in order to show engaging rib 227 (described below in detail).

As shown in FIG. 16, this connector device includes catheter hub 230 and male connector 210 connected thereto. This connector device has a main characteristic in a connection structure of hub 230 and male connector 210. Specifically, the leading end of engaging pin 222 (described below in detail) extending outward in a diameter direction on a base end side of hub 230 is connected to a portion of barrel portion 233 of hub 230 through connecting portion 224 in an axis direction.

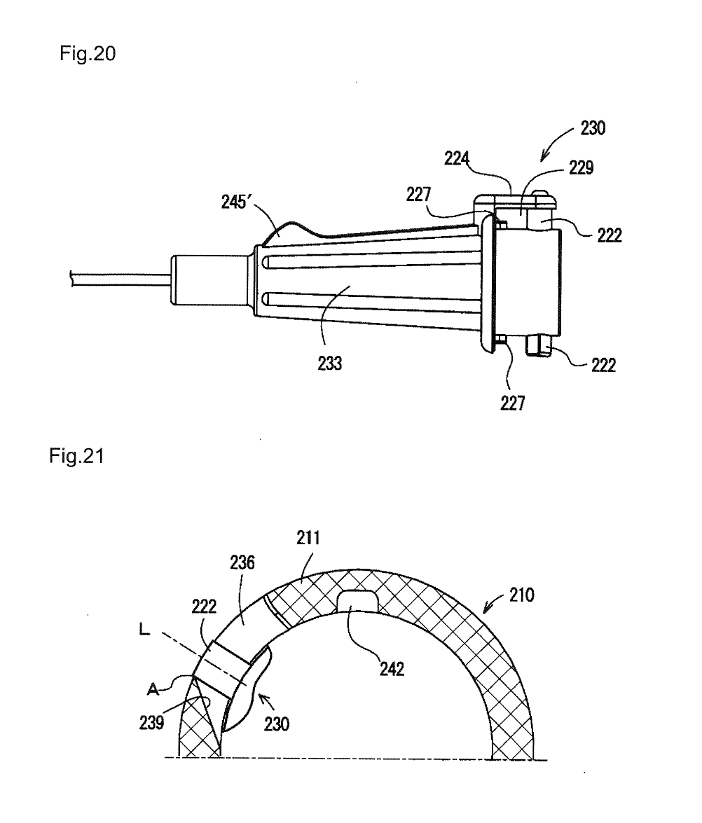

Catheter hub 230 has two engaging pins 222 (see FIG. 20) serving similarly to engaging pins 122 (see FIG. 9) of the above embodiment. Engaging pin 222 is formed to extend outward in the diameter direction on an outer periphery of the hub. One of engaging pins 222 is provided in an upper portion of the outer periphery of the hub and the other engaging pin 222 is provided in a lower portion of the outer periphery of the hub such that engaging pins 222 are placed at intervals of 180°.

As shown in FIG. 20, connecting portion (bridging portion) 224 is formed at the leading end of engaging pin 222 in the upper portion and connecting portion 224 extends in the axis direction (a lateral direction in FIG. 20). The leading end of engaging pin 222 is connected to the portion of barrel portion 233 of hub 230. An engaging hole 229 is formed inside of the connecting portion 224. Engaging tip 235 (see FIG. 17) in a tubular portion of male connector 210 is inserted into engaging hole 229, as later described.

Connecting portion 224 is not limited particularly as long as it connects the leading end of engaging pin 222 to the portion of barrel portion 233 to form engaging hole 229. The extending direction of connecting portion 224 may be in parallel with the axis direction of hub 230 or may be somewhat inclined.

As shown in FIGS. 18 and 20, small rib (protrusion) 227 is formed at a position on a leading end side of the hub within engaging hole 229. Rib 227 is inserted into and engages with recess 242 (see FIG. 17, described below in detail) formed in an inner peripheral face of engaging tip 235 of male connector 210 in the connection state of the connector. As shown in FIG. 20, similar rib 227 is also formed on a lower side of hub 230.

Although not limited particularly, four ribs 238 extending in the axis direction are formed on the outer periphery of barrel portion 233 of hub 230 in this example as shown in FIG. 18. Another rib 245 in the axis direction is formed in the upper portion of barrel portion 233. A portion of rib 245 on the leading end side of the hub is formed as protrusion portion 245′ raised outward in the diameter direction. Since such ribs 238 and protrusion portion 245′ are provided, an operator can favorably hold hub 230 such that the hub is not rotated.

Although not shown, check valve 45 (see FIG. 1) is placed within male connector 230 as in the abovementioned embodiments. Naturally, the check valve may be one as shown in FIG. 5 or FIG. 6.

As shown in FIG. 17, male connector 210 has tubular portion 211 surrounding the male lure portion substantially at the center of a longitudinal direction in the present embodiment. Engaging grooves 236, 236 of generally spiral shape are formed on the top and the bottom of tubular portion 211. Each engaging groove 236 may have a shape similar to those of the above embodiments shown in FIG. 11 and the like.

A portion in front of engaging groove 236 in the figure (on a leading end side of the male connector) is shown as engaging tip 235. Recess 242 bored in the diameter direction is formed in the inner peripheral face of engaging tip 235 (see also FIG. 21).

Although not limited particularly, four ribs 218 in the axis direction are formed on a base end side of male connector 210 for facilitating the grasp of the connector as shown in FIG. 17. Four ribs 218 may be formed at positions of approximately 45°, 135°, 225°, and 315° when male connector 210 is viewed from the axis direction, for example. In this case, since two ribs 218 are placed on each of the left and right sides of male connector 210, the connector can be grasped more easily.

Next, the connection of the connector device configured as above will be described.

First, a catheter (see FIG. 16) attached to the leading end of hub 230 is inserted into a patient's blood vessel as in the abovementioned embodiments.

Next, male connector 210 is connected to catheter hub 230. By way of example, while an operator grasps barrel portion 233 of hub 230 by one hand to hold hub 230 and grasps connector 210 by the other hand to insert the male lure portion into hub 230. At this point, male connector 210 is positioned to be somewhat rotated about the axis line so as to allow insertion of engaging pin 222 of male connector 210 into engaging groove 236 of the hub.

As shown in FIG. 20, catheter hub 230 of the present embodiment is asymmetrical vertically with connecting portion 224 and protrusion portion 245′ formed only on the upper side. Since the top and the bottom of catheter hub 230 are easily identified, such a configuration is particularly preferable when the catheter needs to be inserted in a predetermined orientation.

Returning to the description of the connection of the connector device, as male connector 210 is inserted, the guide operation of engaging pin 222 slid within engaging groove 236 screws the overall male connector 210 into hub 230. At the same time, engaging tip 235 (see FIG. 17) of male connector 210 is inserted into engaging hole 229 (see FIG. 20) of the hub.

When engaging pin 222 is moved to near the end of groove 236, rib 227 of catheter hub 230 engages with recess 242 inside engaging tip 235 of male connector 210. A click feeling is obtained when rib 227 fits into recess 242. Since rib 227 engages with recess 242 in this manner, the connection between hub 230 and male connector 210 is not loosened easily.

As described above, catheter hub 230 is connected to male connector 210. According to the configuration of the present embodiment, since connecting portion 224 is formed at the position of engaging pin 222 on the upper side of catheter hub 230, the top and the bottom of catheter hub 230 is easily identified. In addition, since engaging tip 235 of male connector 210 is inserted into and engages with engaging hole 229 formed by connecting portion 224, the hub and the male connector are connected more tightly.

While the example of the present invention has been described, the above connection structure is not limited to the connection between the catheter hub and the male connector but is applicable to various connections including the connection between tubes. In addition, contrary to the above example, engaging groove 236 may be provided for the catheter hub and engaging pin 222 may be provided for the male connector. The number of engaging pins 222 is not limited to two, and single engaging pin 222 may be used.

FIG. 21 is a schematic diagram of male connector 210 in FIG. 16 viewed from the leading end side, and a hatched portion represents an end face of tubular portion 211.

As shown, inner wall 239 of engaging groove 236 along which engaging pin 222 is slid may be formed not as a straight face in the diameter direction (see line L) but as an inclined face relative to line L. In this case, an outer peripheral face of engaging pin 222 is in point contact with inner wall 239 at point A (located on an outer peripheral side of tubular portion 211). When the connection area is small in this manner, engaging pin 222 is favorably slid along inner wall 239, so that the connection or removal of the connector can be performed more smoothly. Such an inclination of the groove may be formed on the entire guide face of groove 236 (136a, see FIG. 11). Naturally, such an inclination of the groove is also applicable to engaging groove 136 shown in FIG. 8 and the like.

### Other Embodiment 2

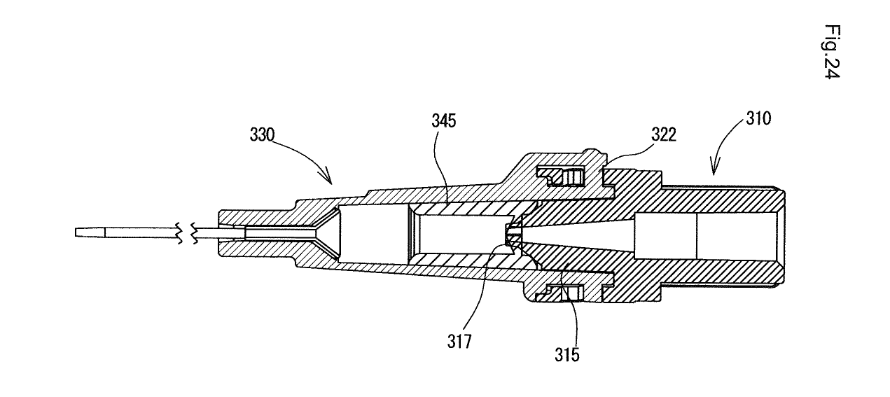

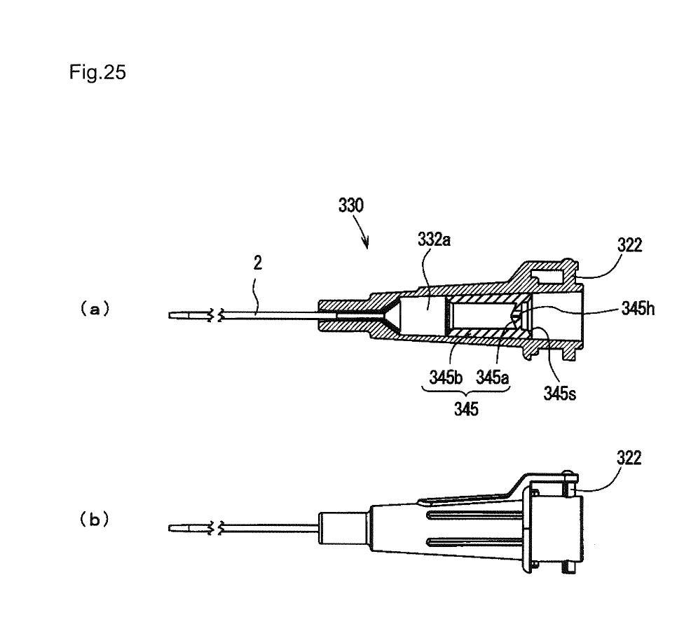

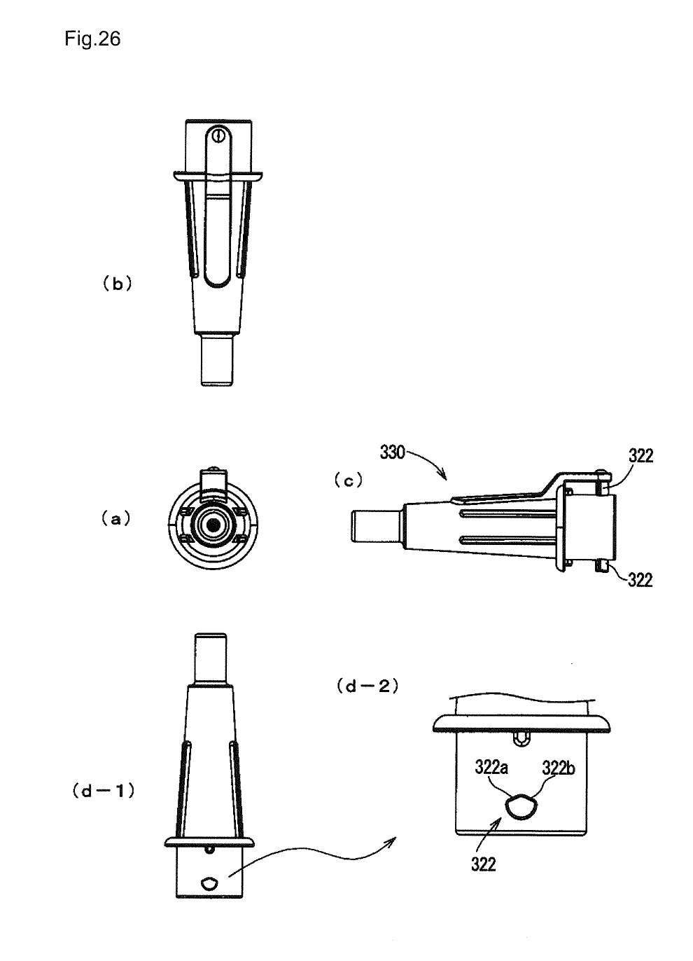

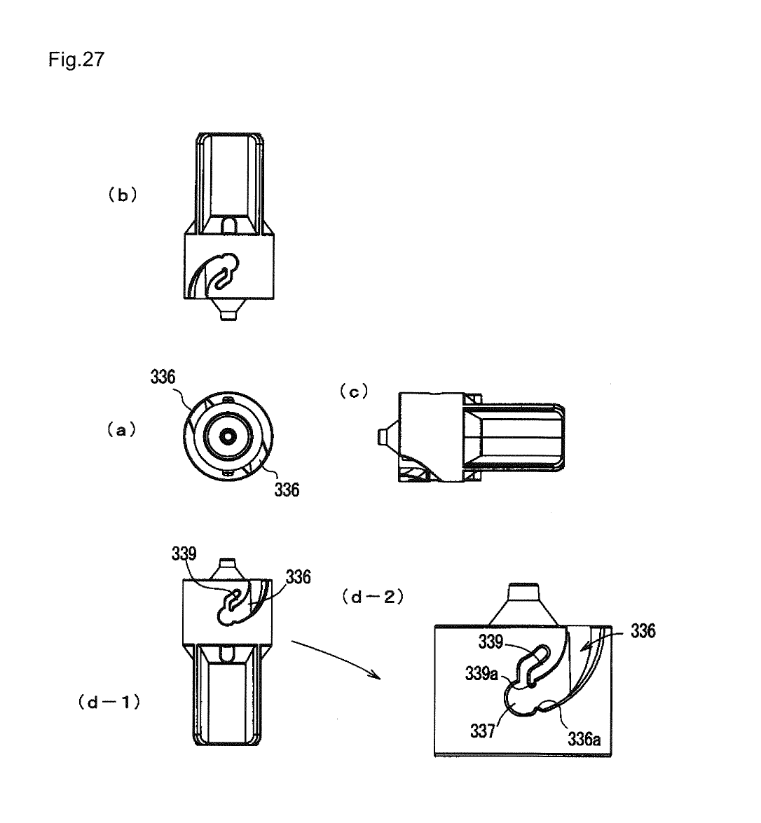

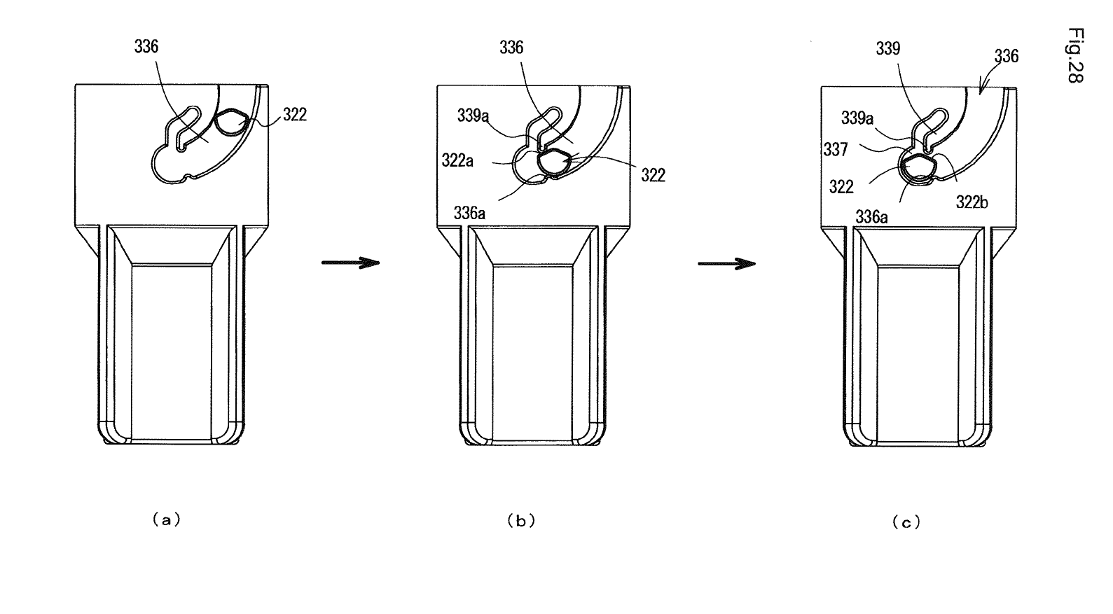

Yet another example of the connection structure of the male connector and the catheter hub will be described with reference to FIGS. 22 to 28. FIG. 22 is a perspective view showing a connector device of the present embodiment before connection.

As shown in FIG. 22, this connector device includes catheter hub 330 and male connector 310 connected thereto. This connector device differs from that in the above embodiments in that the connection structure of hub 330 and male connector 310 is modified and that the shape or the like of check valve 345 within catheter hub 330 is modified (see FIG. 25, later described in detail).

First, check valve 345 will be described with reference to FIGS. 24 and 25.

As shown in FIG. 25(a), check valve 345 has barrel portion 345b of cylindrical shape and valve wall 345a provided near an end portion of barrel portion 345b in a longitudinal direction and extending in a radius direction as in Embodiment 1, and is formed in a bottomed tubular shape as a whole. Valve wall 345a is provided not at the end portion of tubular portion 345b but on a somewhat inner side relative to the end portion (referred to as the “bottomed tubular shape” including such a shape in the present specification).

Valve wall 345a is located on an inlet side of an internal hole of catheter hub 330. Since check valve 345 is placed in this orientation, the size of space 332a upstream from valve wall 345a within male connector 330 is relatively large. With space 332a largely formed, even when blood of a patient reversely flows through catheter 2, it takes a relatively long time to fill space 332a with the blood. Such a configuration is advantageous since the possibility of a leak of blood to the outside is further reduced.

In this example, the thickness of valve wall 345a is not uniform as shown in FIG. 25(a). Valve wall 345a has a section shape of low mountain, with the largest thickness at the center.

As in the above embodiments, single central hole 345h is formed at the center of valve wall 345a (the center viewed from an axis direction). Leading conduit 317 provided at a leading end of male lure portion 315 of the male connector is inserted into central hole 345h (see FIG. 24). Male lure portion 315 is formed to be shorter than that in the above embodiment (see, for example FIG. 1) in accordance with the shape and the placement of check valve 345.

As shown in FIG. 25(a), tapered face 345s is formed at an end portion on an inlet side (left side in FIG. 25(a)) of tubular portion 345b of the check valve. Tapered face 345s, as shown in FIG. 24, is in intimate contact with the outer peripheral face of the leading end of lure portion 315 of the male connector in the state in which the male connector is connected.

According to such a configuration, since the intimate contact ensures sealing in this portion, the possibility of a leak of blood or the like to the outside can be further reduced.

Next, the connection structure will be described with reference to FIGS. 26 to 28.

In the present embodiment, extending portion 339 of cantilever shape is formed at a portion of a peripheral wall of engaging groove 336 of generally spiral shape. Extending portion 339 has a base portion located near an inlet of groove 336 and extends toward the end of the groove. Extending portion 339 is a member for defining the shape of groove 336 and is gently curved so as to provide the generally spiral shape of groove 336 as a whole. Leading end 339a of extending portion 339 protrudes in an axis direction toward the back of the groove (downward in FIG. 27).

As shown in FIG. 27(d-2), protrusion portion 336a protruding in the axis direction toward extending portion 339 is formed at a position opposite to leading end 339a of the extending portion. Protrusion portion 336a and leading end 339a of the extending portion serve to hold engaging pin 322 (see FIG. 28), as later described.

As shown in FIG. 26(d-2), in the present embodiment, the outline shape of engaging pin 322 is modified. Specifically, tapered portions 322a, 322b are formed on both sides of left and right on an upper side (opposite to the extending portion 339) of the outline of engaging pin 322. Two tapered portions 322a, 322b provide a mountain shape for a part of the outline of pin 322.

While FIG. 26(d-2) shows the pin on a lower side of catheter hub 310, pin 322 on an upper side is formed to have a section shape similar to that.

Next, the connection method of the connector in the present embodiment configured as above will be described.

First, a catheter attached to the leading end of hub 330 is inserted into a patient's blood vessel.

Next, male connector 310 is connected to catheter hub 330. Since the basic connection procedure is identical to that in the above embodiment (see FIG. 16), description herein will be made mainly of sliding of engaging pin 322 within groove 336 with reference to FIG. 28. For convenience of illustration and description, FIGS. 28(a) to 28(c) show only engaging pin 322.

First, as male connector (310) is inserted into hub (330), engaging pin 322 is guided and moved toward the end of groove 336 as shown in FIG. 28(a).

Next, when the connector is moved to the state shown in FIG. 28(b), tapered portion 322a of an outer periphery of engaging pin 322 abuts on leading end 339a of the extending portion, and a lower-left portion of the outer periphery of engaging pin 322 abuts on protrusion 336a within the groove.

Then, when the male connector is further screwed into the hub from that state, engaging pin 322 moves on protrusion 336a and extending portion 339 is pushed by engaging pin 322 and is elastically deformed upward.

After a top portion of the mountain formed by tapers 322a and 322b moves beyond leading end 339a of extending portion 339, pin 322 engages at deepest portion 337 of the groove as shown in FIG. 28(c).

In this state, extending portion 339 returns to the original shape and the leading end 339a of extending portion 339 and protrusion 336a locks engaging pin 322 at that position. Specifically, leading end 339a of the extending portion generally abuts on tapered face 322b, and protrusion 336a on the lower side generally abuts on a lower-right portion of the outer peripheral face of engaging pin 322. “Generally abutting” means that it does not necessarily abut.

For removing the connector, male connector 310 may be screwed in the direction reverse to the above direction. The connector can be removed easily without requiring special means or operation for releasing the lock.

According to the configuration of the present embodiment as described above, since extending portion 339 is flexible, a click feeling at the time of locking can be improved, and the reliable lock of the connector can be advantageously checked more easily by the touch.

While some embodiments of the present invention have been described with reference to the drawings, the features described in the embodiments may be combined as appropriate. For example, the placement of check valve 345 as shown in FIG. 25 can achieve the predetermined operation and effect by itself and can be used not only for the connector in FIG. 25 but also for the embodiments as described above.

### DESCRIPTION OF REFERENCE NUMERALS

1, 101 MEDICAL CONNECTOR DEVICE

2 CATHETER

10, 110, 110′, 210, 310 MALE CONNECTOR

15, 115, 215, 315 MALE LURE PORTION

16, 17, 116, 317 LEADING END TUBE

19 PROTRUSION

30, 130, 130′, 230, 330 CATHETER HUB

32 FEMALE LURE FACE

39 ENGAGING SPIRAL

45, 46, 47, 345 CHECK VALVE

45h, 345h CENTRAL HOLE

118 FIN PORTION

119 FRONT END FACE

122, 222, 322 ENGAGING PIN

132 DEPRESSION PORTION

136, 236, 336 ENGAGING GROOVE

138 FIN PORTION

141 PROTRUSION

142, 242 RECESS

211 TUBULAR PORTION

218 RIB

224 CONNECTING PORTION

227, 238, 245 RIB

229 ENGAGING HOLE

337 DEEPEST PORTION

339 EXTENDING PORTION

1. A medical connector device comprising:
a catheter to be inserted into a patient's body;
a catheter hub having a generally tubular shape as a whole and having a base end portion opened and a leading end portion supporting a base portion of the catheter;
a check valve having a central hole with an increasable and reducible diameter and placed within the catheter hub; and
a male connector having a male lure portion to be inserted into the catheter hub from its opened side, and connected to the catheter hub,
wherein the male connector includes a leading conduit at a leading end of the male lure portion, the conduit. being thinner than the male lure portion,
when the male connector is not being connected to the catheter hub, the diameter of the central hole of the check valve is reduced to prevent a backflow of liquid from the catheter, and
when the male connector is being connected to the catheter hub, the leading conduit is inserted into the central hole to increase the diameter of the central hole to allow a flow of liquid and the check valve provides watertight sealing between an outer peripheral face of the leading conduit and an inner peripheral face of the catheter hub.

2. The medical connector device according to claim 1, wherein the leading conduit has a tapered shape.

3. The medical connector device according to claim 1, wherein the male lure portion and the leading conduit are formed of different members.

4. The medical connector device according to claim 1, wherein the check valve further includes a tapered face which guides the leading conduit toward the central hole.

5. The medical connector device according to claim 1, wherein a material of the check valve is rubber.

6. The medical connector device according to claim 1, wherein the check valve has a bottomed tubular shape as a whole, and
the check valve is placed such that its bottom portion is located on an inlet side of an inner hole of the catheter hub.

7. The medical connector device according to claim 1, further comprising a fixing means for fixing the male connector to the catheter hub,
wherein the fixing means includes:
a plurality of engaging spirals formed on an outer peripheral face of the catheter hub; and
a plurality of protrusions formed on the male connector and engageable with the engaging spirals, and
wherein the male connector is screwed into the catheter hub and the male connector is rotated within a range of 180° or smaller about an axis line to engage the protrusion with the engaging spiral in order to connect the male connector to the catheter hub.

8. The medical connector device according to claim 7, wherein an outer peripheral face of the male lure portion contacts intimately with the inner peripheral face of the catheter hub in a state in which the male connector is connected to the catheter hub.

9. The medical connector device according to claim 1, further comprising a fixing means for fixing the male connector to the catheter hub,
wherein the fixing means includes:
a engaging pin formed on one of the male connector and the catheter hub and extending outward in a generally diameter direction; and
a engaging groove of generally spiral shape formed in a tubular portion of the other of the male connector and the catheter hub, the engaging pin being slid within the engaging groove,
wherein the engaging pin engages at the end of the groove to connect the male connector to the catheter hub.

10. The medical connector device according to claim 9, wherein the fixing means comprises an extending portion of cantilever shape formed at a portion of the engaging groove of generally spiral shape,
and wherein,
during the engaging pin is moved toward the end of the groove, the engaging pin abuts on a leading end of the extending portion,
when the engaging pin is further moved toward the end of the groove, the extending portion is pushed by the engaging pin and is elastically bent, and
when the engaging pin is further moved toward the end of the groove, the engaging pin moves beyond the leading end of the extending portion to the end of the groove and engages at that portion.

11. The medical connector device according to claim 9, wherein a protrusion portion which protrudes toward the extending portion is formed at a position opposite to the leading end of the extending portion, and
the leading end of the extending portion and the protrusion portion cooperate to hold the engaging pin.

12. The medical connector device according to claim 9, wherein the fixing means further includes:
a recess formed in an end face of one of the male connector and the catheter hub; and
a protrusion formed on the other of the male connector and the catheter hub and engages in the recess in a state in which the male connector is connected to the catheter hub.

13. The medical connector device according to claim 9, further comprising a bridging portion which connects a leading end of the engaging pin and a portion of one of the male connector and the catheter hub, in an axis direction, to form an engaging hole,
wherein a portion of the tubular portion of the other of the male connector and the catheter hub is inserted into and engages with the engaging hole.

14. The medical connector device according to claim 13, wherein the engaging pin includes two engaging pins formed on an outer periphery of one of the male connector and the catheter hub, and
the bridging portion is provided for only one of the two engaging pins.

15. The medical connector device according to claim 13, wherein the fixing means includes:
a protrusion formed within the engaging hole; and
a recess formed in the tubular portion of the other of the male connector and the catheter hub, the protrusion engaging with the recess.
